(19) United States
(12) Patent Application Publication
(10) Pub. No.: US 2008/0103586 A1
(43) Pub. Date: May 1, 2008

(54) Implant for placing in a blood circulation conduit

(76) Inventors: Mikolaj Styrc, Kopstal (LU); Eric Perouse, Paris (FR)

Correspondence Address: WENDEROTH, LIND & PONACK, L.L.P., 2033 K STREET N. W., SUITE 800, WASHINGTON, DC, 20006-1021, US

(21) Appl. No.: 11/907,503

(22) Filed: Oct. 12, 2007

(30) Foreign Application Priority Data
Oct. 16, 2006 (FR) 06 09054

(51) Int. Cl. A61F 2/06; A61F 2/24

(52) U.S. Cl. 623/1.24

(57) ABSTRACT

This implant (10) comprises a hollow body (30) adapted to be deployed between a compressed configuration and a totally dilated configuration constituting its rest configuration. The hollow body (30) has an internal surface (40) with an axis (X-X′) which delimits a blood circulation passage (42).

The implant (10) comprises at least a constricting flexible threadlike link (68) which is linked to the hollow body at at least two linking points (60A, 60B, 60C) which are spaced angularly round the axis (X-X′) on its internal surface (40). The link (68) can be deployed between a retracted state and a deployed state during deployment of the hollow body (30). The distance separating the linking points (60A, 60B, 60C) in the deployed state of the threadlike link (68) is smaller than the distance separating these points (60A, 60B, 60C) in the totally dilated configuration of the hollow body (30) in the absence of threadlike link (68).

### TECHNICAL FIELD

The present invention relates to an implant for placing in a blood circulation conduit, of the type comprising a hollow body which can be deployed between a compressed configuration and a totally dilated configuration which constitutes its rest configuration, the hollow body having an internal surface with an axis X-X′ delimiting a blood circulation passage.

The invention applies, in particular, to the endovalves formed by a flexible obturator fitted in a tubular endoprosthesis and intended to replace a native cardiac valve.

### BACKGROUND TO THE INVENTION

The heart comprises valves which are located at the outlet of the right ventricle (pulmonary valve) and of the left ventricle (aortic valve).

These valves allow univocal circulation of the blood flow, preventing a reflux of blood following the ventricular contraction.

However, diseases affect the valves. In particular, the valves can suffer from calcification which allows a reflux or a regurgitation toward the ventricle which expelled the blood flow. The problem of regurgitation leads to abnormal dilation of the ventricle which ultimately leads to cardiac failure.

In order to treat this type of disease by surgery, the diseased valve is replaced. It is thus known to implant an endovalve in the opening defined between the lamellae delimiting the diseased valve. This endovalve consists of a tubular endoprosthesis formed by a self-expanding lattice and a flexible obturator produced in a tissue of animal origin. The flexible obturator is permanently fixed in the endoprosthesis.

Endovalves of this type can be implanted endoluminally, and this considerably limits the risks associated with implantation of the valve, in particular in terms of mortality.

Endovalves are not completely satisfactory in some cases. The wall defining the opening through the native valve has cross-sections which vary according to the individual.

In some patients, this opening is circular. In this case, the endoprosthesis supporting the obturator adopts a circular section cylindrical configuration after its deployment in the opening of the native valve, and this allows satisfactory operation of the obturator for a long period of time.

However, the cross-section of the opening of the native valve is elongate or angular in some patients. In this case, the endoprosthesis adopts a corresponding shape when deployed against the wall delimiting the native valve. This shape contributes to deformation of the periphery of the obturator and the lamellae forming it. The obturator and its lamellae cannot operate reliably and satisfactorily for a long period of time if they are partially deformed. Consequently, the obturator deteriorates rapidly, necessitating replacement of the endovalve.

### SUMMARY OF THE INVENTION

An object of the invention is to obtain an implantable endovalve as a replacement for a native valve, the obturator of which operates reliably and tightly for a long period of time, whatever the morphology of the patient.

The present invention accordingly relates to an endoprosthesis of the aforementioned type wherein the implant comprises at least a flexible threadlike link for local constriction of the hollow body permanently fitted on the hollow body, the or each link being linked to the hollow body at at least two linking points angularly spaced round the axis X-X′ on the hollow body, the or each link being adapted to be deployed between a retracted state and a deployed state during the deployment of the hollow body, the distance separating the two linking points in the deployed state of the link being smaller than the distance separating these points in the totally dilated configuration of the hollow body in the absence of threadlike link.

The endoprosthesis according to the invention can comprise one or more of the following features taken in isolation or in any technically feasible combination:
it comprises a flexible obturator which is attached to the internal surface, the linking points being located in a region of the internal surface opposite or in the vicinity of the obturator;
at least a threadlike link is a tension line tensioned linearly through the circulation passage in its deployed state between two linking points on the hollow body;
it comprises at least three substantially coplanar tension bands, the tension lines defining, in their deployed state, a closed polygon in the circulation passage;
the obturator is fixed to at least a circumference passing through the vertices of the polygon;
it comprises at least two intersecting tension lines placed in the circulation passage in their deployed state;
the obturator comprises a plurality of deformable flexible pockets in the internal passage, at least two intersecting tension lines being placed in each pocket;
at least a threadlike link is a constricting peripheral link engaged in the hollow body over a periphery of the hollow body;
the constricting peripheral link comprises a ring which projects from the hollow body;
the or each threadlike link delimits, on the internal surface, at least one region for restricted deployment of the hollow body and at least one region for free deployment of the hollow body.

### BRIEF DESCRIPTION OF THE DRAWINGS

The invention will be understood better on reading the following description which is given merely by way of example with reference to the accompanying drawings in which:

FIG. 1 is a perspective schematic view of a first endovalve according to the invention in which the endoprosthesis occupies a controlled dilation configuration;

FIG. 2 is a plan view of the endovalve from FIG. 1;

FIG. 3 is a partial side view of the endovalve from FIG. 1 implanted in the coronary sinus of a first patient as a replacement for a native valve;

FIG. 4 is a view similar to FIG. 3 for the coronary sinus of a second patient;

FIG. 5 is a view similar to FIG. 1 of a variation of endovalve according to the invention;

FIG. 6 is a view similar to FIG. 2 of a further variation of endovalve according to the invention.

### DESCRIPTION OF PREFERRED EMBODIMENTS

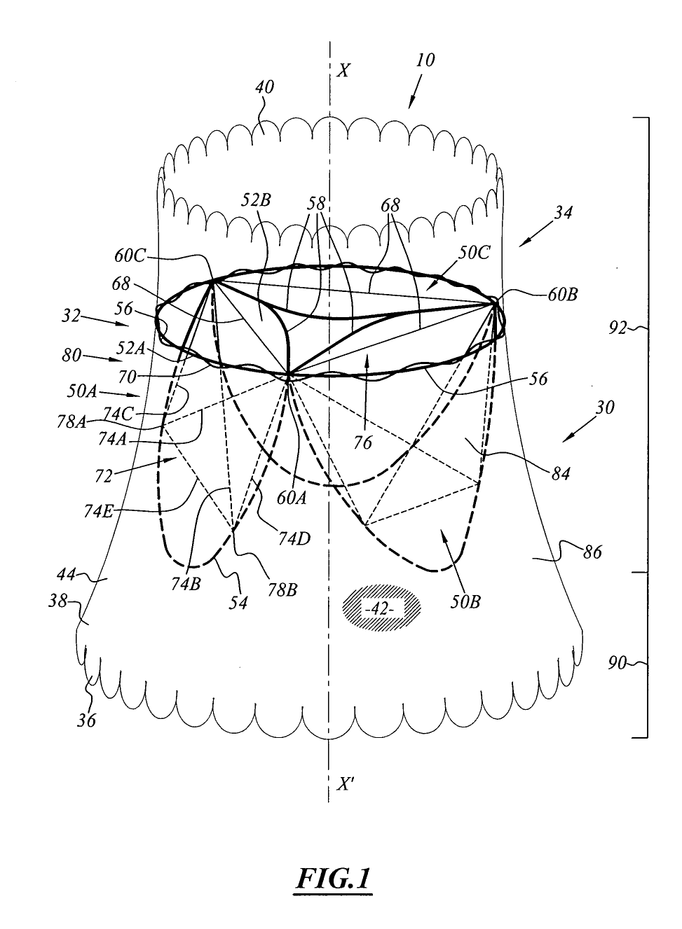

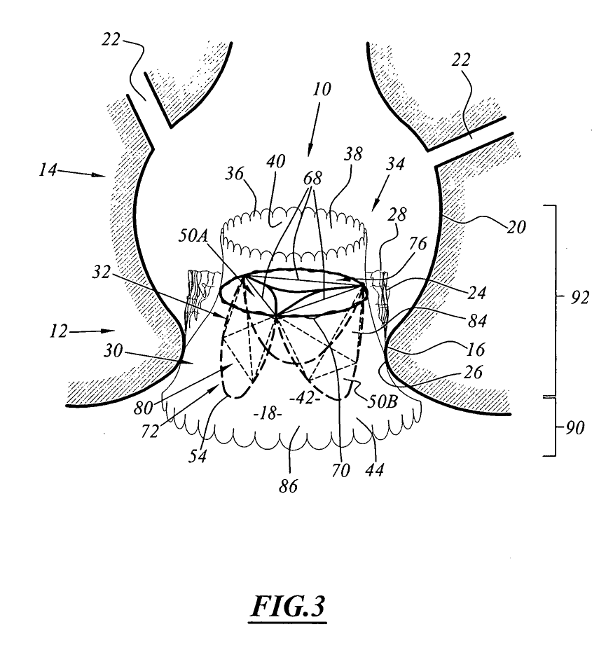

A first implant 10 according to the invention is shown in FIG. 1 to 4. This implant 10 is an endovalve for replacing a deficient native valve 12 shown in FIG. 3 in the coronary sinus 14.

The coronary sinus 14 is defined inside a wall having a narrow part 16 defining a blood circulation opening 18 and a flared region 20 in which the coronary arteries 22 open. The narrow part 16 forms the seat of the native valve 12.

The native valve 12 comprises lamellae 24 having a lower edge 26 articulated to the narrow part 16 and a free upper edge 28 extending in the sinus 14 opposite the flared wall 20.

Referring to FIG. 1, the implant 10 comprises a radially deployable support endoprosthesis 30 and a flexible obturator 32 which is permanently fixed in the endoprosthesis 30.

According to the invention, the implant 10 further comprises a unit 34 for controlling the deployment of the endoprosthesis 30 in the region carrying the obturator 32.

The endoprosthesis 30 is formed, for example, by a tubular lattice 36 of interlaced threads immersed in a liquid-tight extensible film 38 such as an elastomer.

The lattice 36 consists, for example, of stainless steel having resilient properties based on a shape-memory metal or based on a flexible polymeric fibre so that the endoprosthesis 30 is self-expanding.

An endoprosthesis of this type, when used alone, is currently designated by the English term “stent”.

As known per se, the endoprosthesis 30 is capable of deforming spontaneously from a compressed configuration, in which it has a small diameter, to a dilation configuration controlled by the control unit 34 in which it has a greater diameter. If it is not equipped with the control unit 34, the endoprosthesis 30 is also capable of deforming beyond its controlled dilation configuration to a totally dilated configuration constituting its rest state.

The endoprosthesis 30 defines, round an axis X-X′, an internal surface 40 delimiting a central blood circulation passage 42 and an external surface 44 which is to be placed, in part, against the narrow part 16 as will be seen hereinafter.

The obturator 32 comprises three adjacent flexible pockets 50A, 50B, 50C which are permanently fixed in the endoprosthesis 30 and distributed angularly round the axis X-X′.

In the example shown in FIG. 1, each pocket 50A, 50B, 50C comprises an external membrane 52A and an internal membrane 52B of substantially parabolic shape which are connected to one another and to the internal surface 40 along a seam 54.

Each membrane 52A, 52B is formed by a film of polymer or a layer of organic film such as calf's pericardium or a native pig's valve.

The external membrane 52A is delimited at the bottom by the parabolic seam 54. It is delimited at the top by an upper edge 56 of circular contour fixed to the internal surface 40 along a section perpendicular to the axis X-X′ of this surface 40. The external membrane 52A is therefore held against the internal surface 40.

The internal membrane 52B has a shape similar to that of the external membrane 52A. It extends opposite the membrane 52A along the seam line 54. It has a free upper edge 58 which is substantially coplanar with the upper edge 56 of the internal membrane 52A. The length of the edge 58 of the membrane 52B is greater than the upper edge 56 of the external membrane 52A.

The internal membrane 52B is deformable between a position radially spaced from the axis X-X′ in which its upper edge 58 is located in the vicinity of the upper edge 56 of the external membrane 52A and a position radially close to the axis X-X′ in which the upper edge 58 is placed at a distance from the upper edge 56 of the membrane 52A.

The pockets 50A, 50B, 50C are distributed angularly round the axis X-X′ adjacently to one another.

Therefore, each lateral end of an upper edge 56, 58 of each pocket 50A, 50B, 50C is fixed to the internal surface 40 at the same fixing point 60A, 60B, 60C as the lateral end of the upper edges 56, 58 of an adjacent pocket 50A, 50B, 50C. The pockets 50A, 50B, 50C therefore have three fixing points 60A, 60B, 60C which are common to two adjacent pockets. The fixing points 60A, 60B, 60C are distributed angularly over the internal surface 40.

The common fixing points 60A, 60B, 60C are located in a plane substantially perpendicular to the axis X-X′ and form a substantially equilateral triangle in this plane when the endoprosthesis 30 is deployed, as will be seen hereinafter.

The obturator 32 can be deformed between a position for closing the passage 42 and a position for clearing the passage 42.

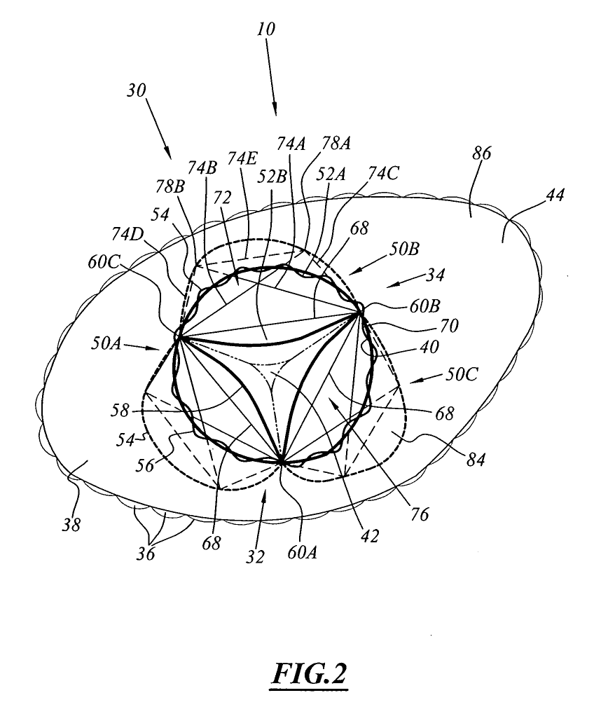

In the closure position shown in broken lines in FIG. 2, the internal membranes 52B occupy all their positions close to the axis X-X′. The free upper edges 58 of the external membranes 52B are coupled two-by-two-by-half, with the exception of a central opening of very small diameter in the vicinity of the axis X-X′.

The passage 42 is therefore substantially closed by the pockets 50A, 50B, 50C which occupy a maximum cross-section in the passage 42.

In the release position, the internal membranes 52B occupy their positions radially spaced from the axis X-X′. The upper edges 58 of the internal membranes 52B are located in the vicinity of the upper edges 56 of the external membranes 52A. The edges 58 are therefore spaced from one another in the passage 42.

The pockets 50A, 50B, 50C., in the release position, occupy a minimum cross-section in the passage 42 and therefore delimit a central opening of large cross-section. The unit 34 for controlling the deployment of the endoprosthesis 30 comprises three upper tension lines 68 adapted to be deployed between the fixing points 60A, 60B, 60C, a peripheral constricting link 70 extending round the upper tension lines 68 and a framework 72 of deployable lower tension lines 74 disposed in each pocket 50A, 50B, 50C.

The upper tension lines 68 are formed by flexible threads produced, for example, on the basis of Nitinol, stainless steel or from a polymer such as mono-filament or multi-filament polyester fibre or an expanded or non-expanded PTFE thread.

The tension lines 68 can be deformed from a retracted state to a deployed state under tension during the radial deployment of the endoprosthesis 30.

Each upper tension line 68 is fixed at its ends between two fixing points 60A, 60B, 60C of the upper edges 56, 58 of the membranes 52A, 52B. Each fixing point 60A, 60B, 60C is therefore linked to two other adjacent fixing points 60A, 60B, 60C via a respective tension line 68 extending through the passage 42. In the example, the fixing points 60A, 60B, 60C therefore form linking points between the tension lines 68 and the endoprosthesis 30.

In the retracted state, each upper tension line 68 is folded on itself for example in the manner of an accordion. The distance between the fixing points 60A, 60B, 60C and therefore the ends of the tension line 68 is smaller than the length of the tension line 68.

On the other hand, when the tension line 68 occupies its deployed state, its ends are located at a distance equal to the length of the tension lines 68. The tension lines 68 are thus tensioned linearly between the fixing points 60A, 60B, 60C.

The length of the tension lines 68 is selected so as to limit the lateral extension of the pockets 50A, 50B, 50C during deployment of the endoprosthesis 30. The distance separating the fixing points 60A, 60B, 60C where the pockets 50A, 50B, 50C are fixed to the internal surface 40 of the endoprosthesis 30 is limited by the upper tension lines 68 and this prevents the membranes 52A, 52B forming the pockets 50A, 50B, 50C from being substantially stretched.

Whatever the circumstances, the distance separating two fixing points 60A, 60B, 60C in the deployed state of each tension line 68 is smaller than the distance separating these two points 60A, 60B, 60C in the totally dilated configuration of the endoprosthesis, in the absence of tension lines 68.

The tension lines 68 in their deployed states thus form a substantially equilateral triangle 76 for controlling the radial deployment of the endoprosthesis 30 along the upper portion of the obturator 32, having the fixing points 60A, 60B, 60C as a vertex.

The peripheral constricting link 70 is engaged in the lattice 36 of the endoprosthesis 30 of a circumference round the axis X-X′. In this example, the link interconnects the fixing points 60A, 60B, 60C along the internal surface 40.

The link 70 therefore defines a plurality of points linking the endoprosthesis 30 which extend over the circumference.

The constricting link 70 can be deployed between a retracted state and a state deployed under tension during deployment of the endoprosthesis 30. In the deployed state, the link 70 has a circular contour in which the triangle 76 is inscribed.

The cross-section of this circle is smaller than the cross-section of the endoprosthesis 30 in its totally dilated configuration in the absence of tension lines 68 and constricting threads 70.

The link 70 therefore limits the radial deployment of the endoprosthesis 30 between each pair of fixing points 60A, 60B, 60C.

The link 70 and the three tension lines 68 therefore constrain the endoprosthesis 30 to adopt a substantially circular cross-section in the plane defined by the fixing points 60A, 60B, 60C during its deployment.

Each framework 72 is placed in a pocket 50A, 50B, 50C between the membranes 52A, 52B, under the upper tension lines 68.

Each framework 72 defines a vertical trapezium 80 comprising two intersecting tension lines 74A, 74B, two lateral tension lines 74C, 74D vertically connecting the intersecting tension lines 74A, 74B, and one base tension line 74E.

Each intersecting tension line 74A, 74B extends between an upper fixing point 60A, 60B, 60C of the upper edges 26, 28 and a lower fixing point 78A, 78B located on the seam 54 opposite this upper fixing point 60A, 60B, 60C relative to a median axial plane of the pocket 50A, 50B, 50C. The intersecting tension lines 74A, 74B form the diagonals of the trapezium 80.

The lateral tension lines 74C, 74D each extend between an upper fixing point 60A, 60B, 60C and the lower fixing point 78A, 78B located on the same side of the pocket relative to a median axial plane of the pocket 50A, 50B, 50C.

The base tension line 74E connects the lower fixing points 78A, 78B substantially in parallel with the upper tension line 68.

Each lower tension line 74A to 74E, similarly to the upper tension line 68, is produced on the basis of a deformable thread. Each tension line 74A to 74E is thus deformable between a retracted state and a linearly tensioned deployed state.

When the endoprosthesis 30 is deployed, the distance separating the fixing points 60A, 60B, 78A, 78B of each tension line 74A to 74E in the linearly tensioned deployed state is less than the distance which would separate these points 60A, 60B, 78A, 78B in the absence of lower tension lines 74A to 74E.

The control unit 34 comprising the upper tension lines 68, the lower tension lines 74A to 74E and the constricting link 70 therefore define on the internal surface 40 of the endoprosthesis 30 a restricted deployment region 84 located opposite the external membranes 52A in which the radial deployment of the endoprosthesis to its totally dilated configuration is limited by the tensioning of the tension lines 68, 64A to 64E and of the link 70.

The unit 34 also delimits, on its internal surface 40, a free deployment region 86 located between the pockets 50A, 50B, 50C and below the pockets 50A, 50B, 50C in which the deployment of the endoprosthesis 30 to its totally dilated configuration is free.

The operation of the first implant 10 according to the invention will now be described.

The implant 10 is initially loaded into a sheath (not shown) for holding the endoprosthesis 30 radially in its compressed configuration.

In this configuration, the fixing points 60A, 60B, 60C are disposed in the vicinity of one another and of the axis X-X′. Similarly, the parabolic seams 54 have branches which are brought towards one another from their vertex so that the lower fixing points 78A, 78B are placed in the vicinity of one another.

In this configuration, the upper tension lines 68, the lower tension lines 74A to 74E and the constricting link 70 all occupy their retracted states.

The sheath containing the implant 10 is then brought into the coronary sinus 14 opposite the narrow part 16 and the lamellae 24 of the native valve 12. It is engaged through the opening 18 in the native valve 12. The sheath is then withdrawn while holding the implant 10 in position, causing the extraction of the implant 10 from the sheath and the gradual radial deployment of the endoprosthesis 30.

Once the sheath has been completely withdrawn, the lower portion 90 of the endoprosthesis 30 located in the free deployment region 86 below the obturator 32 is completely deployed. It comes into contact with the narrow part 16 at the inlet of the coronary sinus 14 so as to effectively fix the implant 10 on the seat of the native valve 12.

In addition, the radial deployment of the upper portion 92 of the endoprosthesis 30 bearing on the obturator 32 is limited by the control unit 34.

For this purpose, during this deployment the upper tension lines 68 are tensioned so as to limit the distance between the fixing points 60A, 60B, 60C of the upper edges of the pockets 50A, 50B, 50C of the obturator 32 and form the substantially equilateral deployment triangle 76.

Similarly, the constricting link 70 is tensioned round a periphery of the endoprosthesis 36 which passes through the fixing points 60A, 60B, 60C so as to limit the radial extension of the endoprosthesis 30 between these points.

The upper edges 56, 58 of the membranes 52A, 52B are therefore substantially contained in a circle, and this prevents harmful deformation of the membranes 52A, 52B.

Similarly, the lower tension lines 74A to 74E are tensioned so as to form a trapezium 80. The lateral and radial extension of the pockets is therefore limited by the maximum dimensions of the trapezium 80 imposed by the length of the tension lines 74A to 74D.

During deployment of the endoprosthesis 30, the respective pockets 50A, 50B, 50C pivot radially away from the axis X-X′ round transverse axes passing through the tension lines 68.

The endoprosthesis 30 therefore occupies its dilation configuration which is controlled by the control unit 34.

The obturator 32 is thus held in a substantially truncated cone-shaped controlled volume, which allows appropriate reliable operation for an extended period of time.

In addition, the constriction of the endoprosthesis 30 in the region 86 located round the obturator 32 limits the risk of application of the external surface 44 of the endoprosthesis against the openings of the coronary arteries 22, in particular in the case of patients having coronary arteries 22 which open opposite the lamellae 24 of the native valve 12 in the coronary sinus 14.

Control of the deployment of the endoprosthesis 30 by the unit 34 therefore also allows a coronary perfusion to be maintained after deployment of the endoprosthesis 30.

The obturator 32 is confined in a volume which is limited by the controlled deployment of the upper portion 92 of the endoprosthesis 30, whatever the morphology of the patient.

Thus, the coronary sinus 14 of the patient shown in FIG. 3 has an opening 18 of native valve 12 with a radial dimension which is greater than the maximum radial dimension of the triangle 76. Once the endoprosthesis 30 has been deployed, the lower portion 90 of the endoprosthesis 30 has a radial dimension round the axis X-X′ which is greater than that of the upper portion 92 opposite the obturator 32.

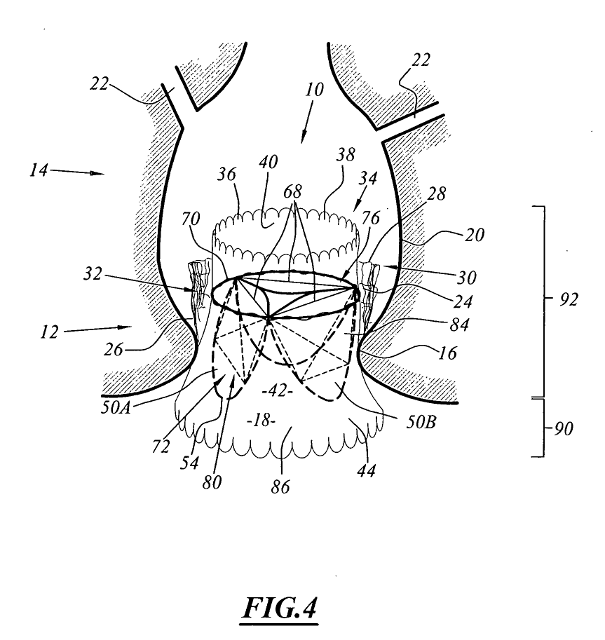

In the example shown in FIG. 4, the narrow part 16 delimits an opening 18 of small diameter. In this case, however, the deployment of the endoprosthesis 30 remains controlled in the upper portion 92 carrying the obturator 32.

In all cases, the lower portion 90 of the endoprosthesis 30 is free to occupy any cross-section, for example an elongate elliptical cross-section as shown in FIG. 2, while maintaining a substantially circular cross-section in the upper portion 92 carrying the obturator 32.

In a variation, the obturator 32 has no external membrane 52A.

Each pocket 50A, 50B, 50C is delimited externally at a distance from the axis X-X′ by the internal surface 40 of the endoprosthesis 30 which is possibly coated with the film 38.

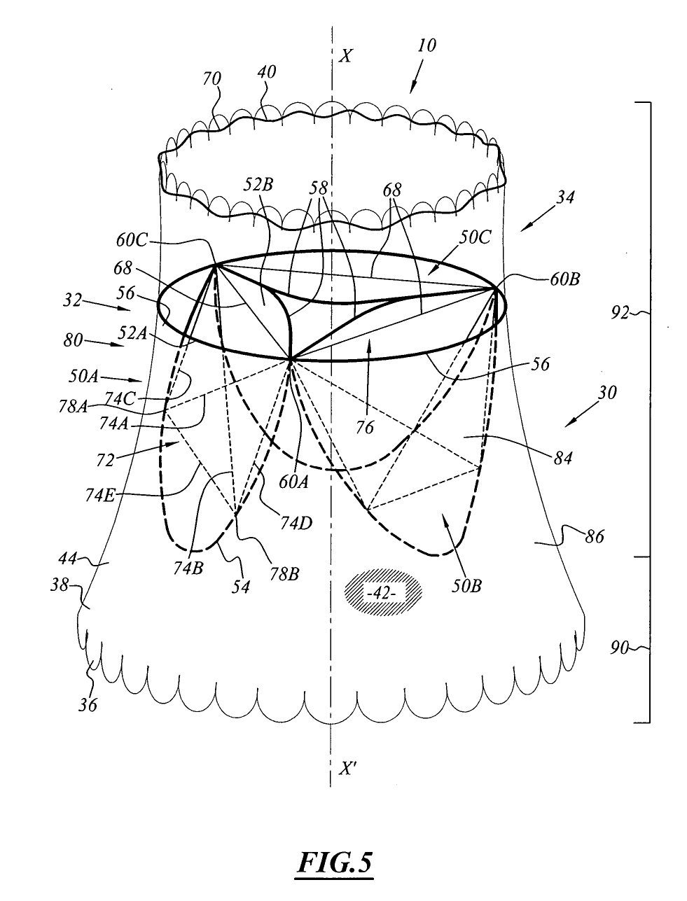

In the variation shown in FIG. 5, the constricting peripheral link 70 is engaged along a circumference of the endoprosthesis 30 which is axially offset toward an upper end of the endoprosthesis 30 relative to the fixing points 60A, 60B, 60C of the free upper edges 58 of the membranes 52A, 52B of the obturator 32.

In another variation (not shown), the points linking the ends of each tension line 68 are axially offset toward an upper end of the prosthesis relative to the common fixing points 60A, 60B, 60C of the free edges 58 of the membranes 52A, 52B of the obturator 32.

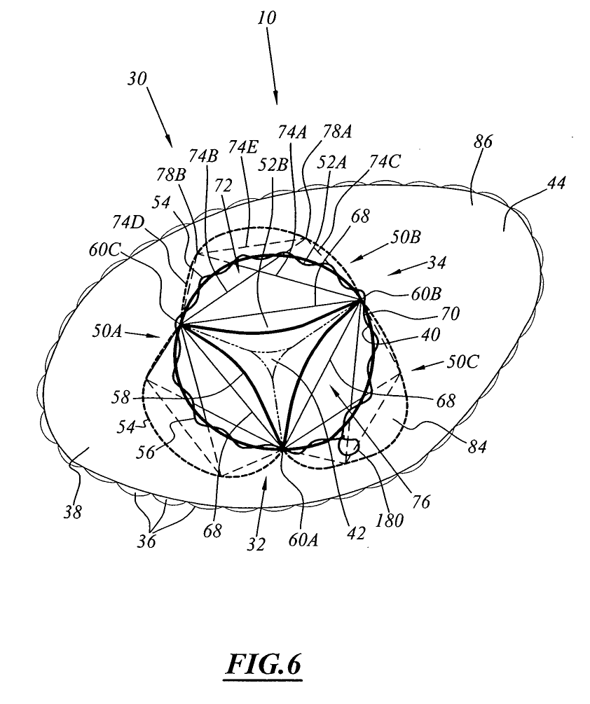

In the variation shown in FIG. 6, the peripheral link 70 is equipped with an external ring 180 which projects externally from the external surface 44 of the endoprosthesis 30. The ring 180 can be gripped by a cross-shaped tool so as to bring about the radial constriction of the endoprosthesis round a circumference.

More generally, the control unit 34 may be devoid of tension lines 68, 74A to 74E which can be deployed through the passage 42 and may comprise a constricting peripheral link 70. Similarly, the control unit 34 may be devoid of constricting peripheral link 70 and comprise tension lines 68, 74A to 74E which can be deployed through the passage 42.

The control unit 34 may also comprise only the tension lines 68.

1. Implant for placing in a blood circulation conduit, of the type comprising:
a hollow body which can be deployed between a compressed configuration and a totally dilated configuration which constitutes its rest configuration, the hollow body having an internal surface with an axis delimiting a blood circulation passage; the hollow body being a tubular endoprosthesis;
wherein the implant comprises at least a flexible threadlike link for local constriction of the hollow body permanently fitted on the hollow body, the or each link being linked to the hollow body at at least two linking points angularly spaced round the axis on the hollow body, the or each link being adapted to be deployed between a retracted state and a deployed state during the deployment of the hollow body, the or each link, in the deployed state, limiting the radial deployment of the endoprosthesis in a controlled dilation configuration, the distance separating the two linking points in the deployed state of the link being smaller than the distance separating these points in the totally dilated configuration of the hollow body in the absence of threadlike link.

2. Implant according to claim 2, wherein it comprises a flexible obturator attached to the internal surface, the linking points being located in a region of the internal surface opposite or in the vicinity of the obturator.

3. Implant according to claim 1, wherein at least a threadlike link is a tension line tensioned linearly through the circulation passage in its deployed state between two linking points on the hollow body.

4. Implant according to claim 3, wherein it comprises at least three substantially coplanar tension bands, the tension lines defining, in their deployed state, a closed polygon in the circulation passage.

5. Implant according to claim 4, wherein the obturator is fixed to at least a circumference passing through the vertices of the polygon.

6. Implant according to claim 3, wherein it comprises at least two intersecting tension lines placed in the circulation passage in their deployed state.

7. Implant according to claim 6, wherein the obturator comprises a plurality of deformable flexible pockets in the internal passage, at least two intersecting tension lines being placed in each pocket.

8. Implant according to anyone of claims 1 to 7, wherein at least a threadlike link is a constricting peripheral link engaged in the hollow body over a periphery of the hollow body.

9. Implant according to claim 8, wherein the constricting peripheral link comprises a ring which projects from the hollow body.

10. Implant according to anyone of claims 1 to 7, wherein the or each threadlike link delimits, on the internal surface, at least one region for restricted deployment of the hollow body and at least one region for free deployment of the hollow body.
